(19) United States
(12) Patent Application Publication
(10) Pub. No.: US 2026/0060854 A1
(43) Pub. Date: Mar. 5, 2026

(54) THIN TEST STRIP, TEST STRIP ROLL, AND APPLICATION OF THIN TEST STRIP

(71) Applicants: China National Pulp and Paper Research Institute Co., Ltd., Beijing City (CN); Sinolight Specialty Fiber Materials Co., Ltd., Langfang City (CN)

(72) Inventors: Fan DU, Beijing City (CN); Lijun LI, Beijing City (CN); Xue ZHANG, Beijing City (CN); Cui MAO, Beijing City (CN); Xuemei CHEN, Beijing City (CN); Guangzhao YANG, Beijing City (CN); Yue ZHANG, Beijing City (CN); Tao ZHAO, Beijing City (CN); Chao TIAN, Beijing City (CN); Guihua XIAO, Beijing City (CN); Xiu DU, Langfang City (CN)

(21) Appl. No.: 19/228,690

(22) Filed: Jun. 4, 2025

(30) Foreign Application Priority Data
Sep. 5, 2024 (CN) 202422180153.0
Oct. 25, 2024 (CN) 202411495420.1

(51) Int. Cl. A61F 13/84; A61F 13/15; G01N 21/77; G01N 21/78; G01N 33/52

(52) U.S. Cl. CPC A61F 13/84; G01N 21/78; G01N 33/528; A61F 2013/15186; A61F 2013/8473; G01N 2021/7759

(57) ABSTRACT

The present disclosure relates to the technical field of tests, and in particular, to a thin test strip, a test strip roll, and an application of the thin test strip. The thin test strip includes: a waterproof top layer, a composite layer, and a waterproof bottom layer in sequence from top to bottom, where the composite layer includes a sample collection pad, a colloidal gold conjugate pad, a color developing membrane, and a sample absorption pad, the waterproof top layer does not cover the sample collection pad and completely covers the colloidal gold conjugate pad, the color developing membrane, and the sample absorption pad, and the composite layer is I-shaped. The waterproof top layer and the waterproof bottom layer are made from polyethylene terephthalate (PET) waterproof transparent membranes.

### CROSS-REFERENCE TO RELATED APPLICATIONS

The present application claims the priority to Application No. 202422180153.0 filed on Sep. 5, 2024 and entitled “HEALTH TEST STRIP FOR SANITARY PRODUCT” and the priority to Application No. 202411495420.1 filed on Oct. 25, 2024 and entitled “TEST STRIP, SANITARY PRODUCT, AND APPLICATION”, which are incorporated herein in their entireties by reference.

### TECHNICAL FIELD

The present disclosure relates to the technical field of tests, and in particular, to a thin test strip, a test strip roll, and an application of the thin test strip.

### BACKGROUND

Test strips are widely used in life and experiments, and can be used for semi-quantitative testing of biochemical indexes quickly and simply. A bottom layer of a conventional colloidal gold test strip is a rigid plastic plate (for example, a polyethylene terephthalate (PET)/polyethylene(PE)/polyvinyl chloride (PVC) bottom plate), and a sample pad, a colloidal gold conjugate pad, a nitrocellulose (NC) membrane, and an absorbent pad are sequentially lap-jointed on the rigid plastic plate from left to right. In recent years, with the development of the medical level and improvement of health awareness, the demand for portable testing of physiological indicators is increasing day by day, and ever-increasing sanitary products with the health monitoring function become available in specific forms of combining the test strips into the sanitary products such as sanitary pads and sanitary napkins.

For example, the patent with publication number of CN219289926U discloses a sanitary pad for a pregnancy test and a sexually transmitted disease test. The sanitary pad includes a sanitary pad body, where the sanitary pad body includes a soft cotton surface layer, a pure cotton absorption layer, a breathable bottom membrane layer, and a release paper layer that are sequentially arranged from top to bottom; a pregnancy test paper set and a sexually transmitted disease test paper set are arranged between the soft cotton surface layer and the pure cotton absorption layer, the two test paper sets are distributed in a length direction of the pad body, each of the two test paper sets includes a plurality of test strips parallel to one another and distributed at intervals, and a length direction of the test strip is consistent with the length direction of the sanitary pad body; and the test strip sequentially includes a sample pad, a colloidal gold conjugate pad, a nitrocellulose membrane, and an absorbent paper from a middle to an end of the sanitary pad body, and ends, closer to and farther from the sample pad, of an upper surface of the nitrocellulose membrane are provided with a test line and a control line respectively. By arranging the test strip under the soft cotton surface layer, the uncomfortable feeling caused by protruding of the test strip is avoided, and the use feeling is better. In addition, a gap is arranged between two adjacent test strips and between two groups of test strips, so as to avoid influence on normal infiltration of urine and secretions that do not enter the sample pad. In a middle, corresponding to the sample pad of each test strip, of the soft cotton surface layer is provided with several absorption holes in a penetrating manner. In addition, the technology further clearly discloses a method for manufacturing the test strip. The method includes: bonding the sample pad, the colloidal gold conjugate pad, the nitrocellulose membrane, and the absorbent paper to the PVC plastic plate once, cutting, with a special knife, the test strip into strips of 0.3 cm×6 cm, adding a desiccant, and performing sealing and independent packaging for later use. Further, as disclosed, when the sanitary pad is manufactured, the layers of the sanitary pad are combined in order blow. The soft cotton surface layer is thermally bonded to a transparent thermoplastic polyurethane (TPU) membrane (not shown), and then the test strip is thermal press laminated followed by the pure cotton absorption layer and the breathable bottom membrane layer, a back adhesive is sprayed, and a release paper layer 5 is finally laminated.

However, the prior art has the following technical problems:

The colloidal gold test strip in the prior art is often used for on-site rapid tests such as clinical diagnosis, environmental monitoring, and food safety testing, and its bottom layer is a rigid PET/PE/PVC plastic bottom plate for the convenience of hold or fixing to a cartridge. As a result, a strong foreign body sensation is caused when the colloidal gold test strip is directly applied to sanitary products.

In order to solve the foreign body sensation, the inventor directly removed the plastic bottom plate, which resulted in a poor test effect. After analysis, the inventor inferred that the poor test effect was caused due to loss of stable support for other parts of the chromatography test strip from the plastic bottom plate. After the plastic bottom plate is removed, a chromatography membrane is likely to deform due to external pressure and bending during the test. Once the chromatography membrane deforms, a chromatography path of a sample in the membrane will change, which will lead to the failure of the sample to react in a test zone and a control zone as expected. Eventually, test results will deviate, and even false positive or negative results will occur.

Despite further studying to narrow the test strip into an elongated I-shaped structure for reducing the foreign body sensation, the inventor still found test inaccuracy and difficult reading outstanding. After analysis and inference, after a wide and thick test strip is directly narrowed, the test strip is likely to bend and deform due to human movement during use, and is hard to restore due to its hard structure. As a result, the sample is hindered from evenly spreading forwards on the parts in the bent zone, and the test accuracy is still affected.

To sum up, it is urgent to develop a test strip structure that balances test accuracy and reduction in foreign body sensation.

### SUMMARY

An objective of the present disclosure is to provide a thin test strip, a test strip roll, and an application of the thin test strip for solving the technical problem of a strong foreign body sensation when an existing test strip is applied to a sanitary product.

In order to achieve the above objective, the present disclosure uses the following technical solution:

A thin test strip includes: a waterproof top layer, a composite layer, and a waterproof bottom layer in sequence from top to bottom, where the composite layer includes a sample collection pad, a colloidal gold conjugate pad, a color developing membrane, and a sample absorption pad, the waterproof top layer does not cover the sample collection pad and completely covers the colloidal gold conjugate pad, the color developing membrane, and the sample absorption pad, and the composite layer is I-shaped.

Preferably, as an improvement, the waterproof top layer and the waterproof bottom layer are made from polyethylene terephthalate (PET) waterproof transparent membranes.

Preferably, as an improvement, a thickness of the waterproof top layer and a thickness of the waterproof bottom layer are 0.03 mm-0.06 mm.

Preferably, as an improvement, a length of the sample collection pad is less than a length of the sample absorption pad.

Preferably, as an improvement, the length of the sample collection pad is 0.3 cm-0.9 cm, and the length of the sample absorption pad is 1.0 cm-1.5 cm.

Preferably, as an improvement, a width of the sample collection pad and a width of the sample absorption pad are 0.5 cm-2 cm.

Preferably, as an improvement, the width of the sample collection pad and the width of the sample absorption pad are equal.

Preferably, as an improvement, a width of the colloidal gold conjugate pad and a width of the color developing membrane are 0.2 cm-0.4 cm.

Preferably, as an improvement, a thickness of the sample collection pad and a thickness of the sample absorption pad are <1 mm.

Preferably, as an improvement, a length of the colloidal gold conjugate pad is 0.2 cm-0.8 cm.

Preferably, as an improvement, a width of the colloidal gold conjugate pad and a width of the color developing membrane are equal.

Preferably, as an improvement, a thickness of the colloidal gold conjugate pad is 0.1 mm-0.5 mm.

Preferably, as an improvement, a length of the color developing membrane is 1.5 cm-2.5 cm.

Preferably, as an improvement, a thickness of the color developing membrane is 0.1 mm-0.5 mm.

Preferably, as an improvement, lengths, lap-jointed with an upper surface of the color developing membrane, of the colloidal gold conjugate pad and the sample absorption pad are 1 mm-3 mm, and a length, lap-jointed with an upper surface of the colloidal gold conjugate pad, of the sample collection pad is 1 mm-2 mm.

Preferably, as an improvement, a material of the sample collection pad is any absorbent material including cellulose, fiberglass, and a polyester fiber, and a material of the sample absorption pad is any absorbent material including the cellulose, the fiberglass, and the polyester fiber.

Preferably, as an improvement, a material of the color developing membrane is any porous material including nitrocellulose or polyvinylidene fluoride, an end, closer to the colloidal gold conjugate pad, of an upper surface of the color developing membrane is provided with a test line T, and an end, farther from the colloidal gold conjugate pad, of the upper surface of the color developing membrane is provided with a control line C.

Preferably, as an improvement, a thickness of the test strip is 0.2 mm-1.2 mm, a length of the paper strip is 3 cm-8 cm, and a width of the test strip is 0.5 cm-2 cm.

The present disclosure further provides a test strip roll. The test strip roll includes a roll formed by mutually connecting the thin test strips.

The present disclosure further provides an application of the thin test strip to a sanitary product, manufacture of a product for monitoring a physiological indicator, or a pet sanitary product.

The disclosure has the beneficial effects:

The thin test strip of this technology changes the foreign body sensation of a traditional test strip, and further ensures test accuracy, even for a test of such a small sample quantity applied to the sanitary pad. Moreover, the thin test strip can be rolled, thus greatly improving processing efficiency of subsequent processing to the sanitary product. The details are as follows.

1. The test strip of this technology adopts a structure that the waterproof top layer, the composite layer, and the waterproof bottom layer are sequentially arranged from top to bottom, and a rigid plastic plate structure at a bottom layer of a conventional colloidal gold test strip is removed, such that the problem of the strong foreign body sensation is solved.

The waterproof layers are used above and below the composite layer to cover the composite layer and connect various areas, such that transfer obstruction due to irreversible deformation can be avoided, and the overall test accuracy can be improved. Moreover, the waterproof top layer and waterproof bottom layer are made from the PET waterproof transparent membranes, such that a user checks color development situation advantageously. In addition, the material is flexible to some extend and connects portions of the composite layer, such that transfer obstruction caused by the irreversible deformation can be further avoided. Further, the thickness of the waterproof top layer and the thickness of the waterproof bottom layer are set as 0.03 mm-0.06 mm. It is found by the inventor through experiments that deformation is likely to occur due to pulling force during processing when the thickness of the waterproof top layer and the thickness of the waterproof bottom layer are less than 0.03 mm, and the strong foreign body sensation occurs when the thickness of the waterproof top layer and the thickness of the waterproof bottom layer are greater than 0.06 mm.

To sum up, the waterproof bottom layer, the waterproof top layer, and design of transparent materials and thickness parameters of the waterproof bottom layer and the waterproof top layer can ensure quality of a final molded products, and can further greatly reduce the foreign body sensation and improve the test accuracy.

In this technology, the composite layer is I-shaped as designed, such that deformation can be further reduced, and accuracy can be improved. Specifically, since the sample absorption pad makes up an upper horizontal part, the colloidal gold conjugate pad and the color developing membrane make up a middle vertical part, and the sample collection pad makes up a lower horizontal part, that is, the sample absorption pad and the sample collection pad serve as the wide widths. The design of the sample absorption pad increases a contact area between the sample absorption pad and the sample, and the sample is easier to transport to the colloidal gold conjugate pad and the color developing membrane. The colloidal gold conjugate pad and the color developing membrane jointly make up a narrow width of the middle vertical part. Because of a narrow lateral width of the middle vertical part, the middle vertical part is not likely to bend and deform downwards basically from a center line to two sides, such that evenly transfer is more likely to occur. Thus, the test accuracy is improved by the I-shaped design.

Due to the I-shaped design of the composite layer, the narrow width part of the middle vertical part made up of the colloidal gold conjugate pad and the color developing membrane can be completely covered with the waterproof top layer and the waterproof bottom layer, such that airtightness of the colloidal gold conjugate pad and the color developing membrane is improved, contact with air is reduced, and a shelf life is prolonged. Moreover, in a process of hold/transporting, the waterproof top layer and the waterproof bottom layer completely covers the color developing membrane, such that scratches are avoided. Moreover, a blank in a narrow zone of the composite layer is actually completely covered with the waterproof top layer and the waterproof bottom layer, and the flexible material can better protect the zone and avoid test accuracy reduction caused by the irreversible deformation.

3. This technology further designs parameters of pad layers in the composite layer, such that the overall test accuracy is improved. Specifically, the width of the colloidal gold conjugate pad and the width of the color developing membrane are equal and 0.2 cm-0.4 cm, and the length of the colloidal gold conjugate pad is designed as 0.2 cm-0.8 cm. Through experiments of the inventor, if the length and the width of the colloidal gold conjugate pad are less than 0.2, an insufficient amount of colloidal gold is caused, color development of the color developing membrane is not obvious, thus affecting reading accuracy. When the length is greater than 0.8 cm, a false positive is likely to occur since too much colloidal gold exists on the pad, and is captured excessively. If the width of the color developing membrane is less than 0.2 cm, a poor color development is caused. However, if the width is greater than 0.4 cm, high cost and a requirement for a great amount of sample liquids are caused.

The length of the sample absorption pad is 1.0 cm-1.5 cm. Through the experiments of the inventor, when the length of the sample absorption pad is less than 1 cm, the too short sample absorption pad weakens “driving force” at a tail end, and the sample cannot completely flow through a test zone of the color developing membrane. When the length of the sample absorption pad is greater than 1.5 cm, the excessively long sample absorption pad enhances an capillary action, and rapid absorption of the sample without sufficient reaction is caused accordingly.

The width of the sample collection pad and the width of the sample absorption pad are 0.5 cm-2 cm. When the width is less than 0.5 cm, the excessively narrow sample collection pad cannot effectively collect and absorb the sample. When the width is greater than 2 cm, an excessively wide sample collection pad causes the sample to be continuously transferred forwards to the colloidal gold pad only if the sample collection pad is completely soaked in the sample, such that a large amount of samples is required. Moreover, the excessively wide sample collection pad/sample absorption pad makes the sample spend too much time in lateral diffusion. In addition, in the case of an excessively great width, an edge of the test strip is likely to bend due to uneven stress, lap joint misalignment of the colloidal gold conjugate pad and the color developing membrane is caused, the sample can not be transferred normally, and the test accuracy is affected accordingly.

More importantly, the length of the sample collection pad in this technology is less than the length of the sample absorption pad, such that an area of the overall sample collection pad is less than an area of the sample absorption pad, the sample can be more easily absorbed to the other side by the sample absorption pad after coming into contact with the sample collection pad, and test efficiency and test accuracy are improved.

To sum up, this technology specially designs the parameters of the composite layer, further improving the test accuracy.

4. In order to balance the stability and the test accuracy of the overall test strip, this technology also specially design a lap joint. Specifically, the lengths, lap-jointed with the upper surface of the color developing membrane, of the colloidal gold conjugate pad and the sample absorption pad are 1 mm-3 mm, and the length, lap-jointed with the upper surface of the colloidal gold conjugate pad, of the sample collection pad is 1 mm-2 mm. If the length, lap-jointed with the upper surface of the colloidal gold conjugate pad, of the sample collection pad is less than 1 mm, the short lap joint leads to discontinuity of a “liquid bridge” between the sample collection pad and the colloidal gold conjugate pad, the sample cannot probably be transferred to the bonding pad smoothly, and the problem that the sample cannot be transferred effectively is likely to occur. In addition, in the process of wear, when the human body moves, the sample collection pad and the colloidal gold conjugate pad are likely to get separated due to the narrow lap joint. However, if such a length is greater than 2 mm, excessive accumulation is likely to occur, and result in “liquid resistance”, a transfer speed of the sample to the test zone is delayed, liquid movement is hindered, and an adsorption effect of the colloidal gold is affected, which is not conducive to guarantee of the test accuracy.

If the lengths, lap-jointed with the upper surface of the color developing membrane, of the colloidal gold conjugate pad and the sample absorption pad are less than 1 mm, a failure of the sample to transfer effectively is likely to occur, resulting in weak or no color development. Moreover, when the product is applied to a wear process, and the human body moves, the lap joint is likely to get separated due to the narrow lap joint. However, if such a length is greater than 3 mm, the excessive accumulation is likely to occur, and result in the “liquid resistance”, and the transfer speed of the sample to the test zone is delayed.

It is worth emphasizing that in this technology, a maximum length, lap-jointed with the upper surface of the colloidal gold conjugate pad, of the sample collection pad is less than the lengths, lap-jointed with the upper surface of the color developing membrane, of the colloidal gold conjugate pad and the sample absorption pad. An objective of this design is to maximize sensitivity and specificity while ensuring a test speed. This technology is especially suitable for a case of a small amount of samples, the sample on the sample collection pad can be rapidly transferred, the sample on the color developing membrane can further fully react, and the overall test accuracy can be further improved.

5. In this technology, the thickness of the colloidal gold conjugate pad is designed as 0.1 mm-0.5 mm, the thickness of the color developing membrane is 0.1 mm-0.5 mm, and the thickness of the sample collection pad and the thickness of the sample absorption pad are <1 mm, such that a thickness of a thickest portion of the overall test strip is controlled as 0.2 mm-1.2 mm. Through design, the length of the colloidal gold conjugate pad is 0.2 cm-0.8 cm, the length of color developing membrane is 1.5 cm-2.5 cm, the length of the sample collection pad is 0.3 cm-0.9 cm, and the length of the sample absorption pad is 1.0 cm-1.5 cm, such that a length of the overall test strip formed through mutual lap joints of these layers is 3 cm-8 cm. The width of the sample collection pad and the width of the sample absorption pad are 0.5 cm-2 cm, such that a maximum width of the overall test strip is 0.5 cm-2 cm.

It is worth emphasizing that the thickness of the test strip is 0.2 mm-1.2 mm, the length of the test strip is 3 cm-8 cm, and the width of the test strip is 0.5 cm-2 cm, thus eliminating the foreign body sensation, and avoiding influence on the overall test accuracy.

6. The existing test strips are independent structures. In upstream and downstream industrial chains, a test strip manufacturer needs to package the test strips separately, resulting in high cost. However, when the test strip is processed into the sanitary product such as the sanitary pad, the test strip needs to be taken out and fixed separately, resulting in low processing efficiency.

Due to the design of the thin test strip in this technology, the test strip can be produced without cutoff. That is, the sample collection pads at one ends of adjacent test strips are arranged in an elongated shape without cutoff, and the sample absorption pads at the other ends are also arranged in an elongated shape without cutoff, such that a plurality of test strips are integrally arranged in an elongated shape, and the elongated shape is formed into the roll. With this arrangement, each roll includes the plurality of test strips, and needs to be packaged with one packaging bag merely, thus greatly saving energy and reducing cost. On the other hand, in a subsequent production process of the sanitary product, the test strip can be used as the roll and dragged to the sanitary product for integrated processing. Compared with the prior art that a single test strip is taken out and fixed, this method can greatly reduce the processing cost and improve the processing efficiency.

To sum up, this technology breaks through the conventional thinking limitation in this field, designs the composite layer in an I-shape, and combines the waterproof layers above and below the composite layer to form a whole, thus obtaining the idea of the thin test strip and arranging the thin test strips in rolls. The thin test strip can reduce the foreign body sensation and further ensure the test accuracy. The test strip roll can reduce upstream and downstream costs, and improve efficiency of subsequent integration and combination with products such as the sanitary product.

### BRIEF DESCRIPTION OF THE DRAWINGS

FIG. 1 is a perspective structural diagram of a thin test strip according to the present disclosure.

FIG. 2 is a structural exploded view of a thin test strip according to the present disclosure.

FIG. 3 is a top view of a thin test strip according to the present disclosure.

FIG. 4 is a right side view of the thin test strip in FIG. 3 before pressing.

FIG. 5 is a top view of mounting the thin test strip of FIG. 1 to a sanitary pad.

FIG. 6 is a schematic structural diagram of a test strip roll.

FIG. 7 is a real picture of FIG. 6.

FIG. 8 is a schematic state diagram of a bent traditional colloidal gold test strip, where a left colloidal gold conjugate pad is separated from a nitrocellulose (NC) membrane and a right sample absorption pad is separated from the NC membrane.

FIG. 9 is a test picture of a bending performance of a thin test strip according to the present disclosure.

FIG. 10 is a waterproof test picture of a thin test strip according to the present disclosure.

FIG. 11 is a top view of a thin test strip (right) of the present disclosure and a traditional colloidal gold test strip (left), from which it can be seen that an area of a sample collection pad of this technology is obviously increased.

FIG. 12 is a humidity test picture of a conventional colloidal gold test strip.

FIG. 13 is a humidity test picture of a thin test strip according to the present disclosure.

### DETAILED DESCRIPTIONS OF THE EMBODIMENTS

Detailed description will be made below with reference to specific embodiments.

Symbols in the accompanying drawings of the description include: 1—waterproof top layer, 2—composite layer, 21—sample collection pad, 22—colloidal gold conjugate pad, 23—color developing membrane, 231—test line T, 232—control line C, 24—sample absorption pad, and 3—waterproof bottom layer.

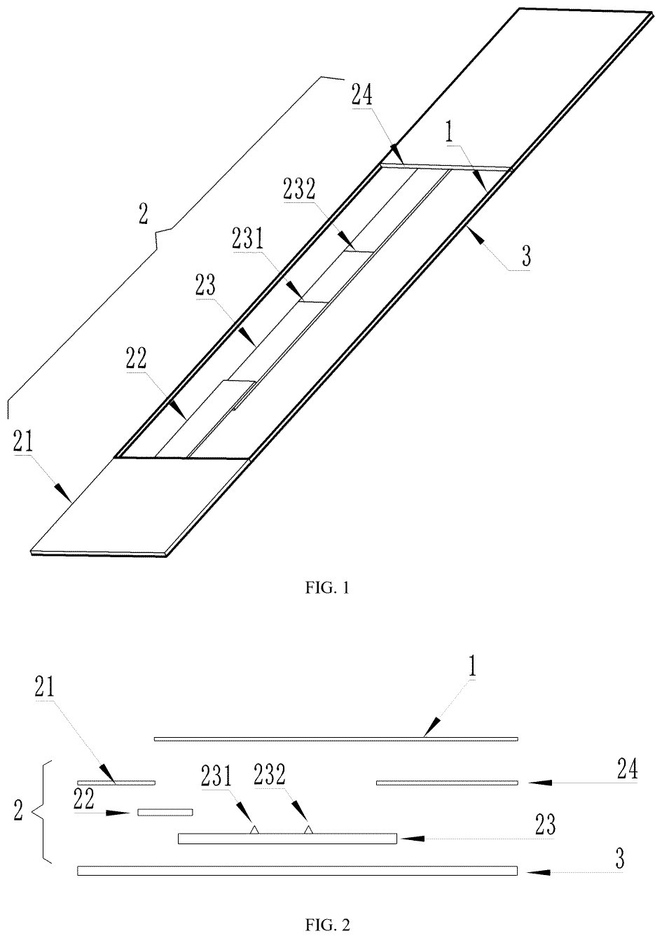

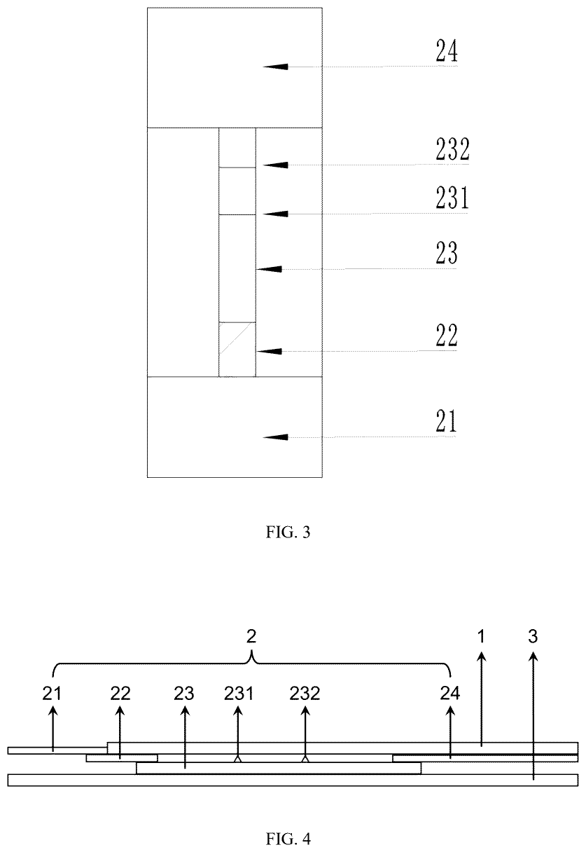

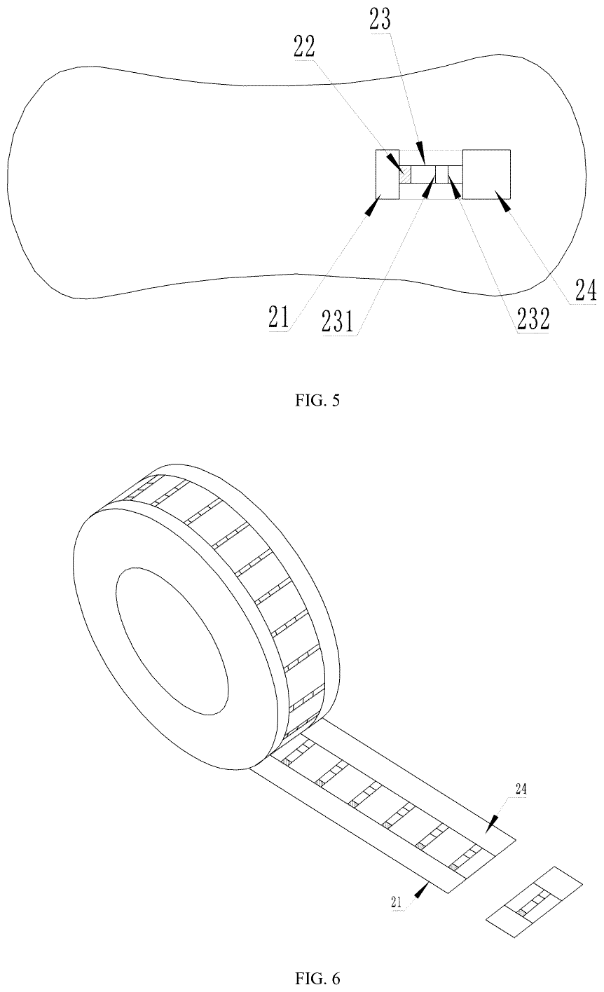

As shown in FIG. 1 to FIG. 13, the present disclosure provides a thin test strip. The thin test strip includes: a waterproof top layer 1, a composite layer 2, and a waterproof bottom layer 3 in sequence from top to bottom, specifically, three vertically stacked layers in which the test strip is laid flat. The composite layer 2 includes a sample collection pad 21, a colloidal gold conjugate pad 22, a color developing membrane 23, and a sample absorption pad 24 that are sequentially lap-jointed from left to right, and are sequentially overlapped arranged partially and connected on the transparent waterproof bottom layer 3, and the transparent waterproof top layer 1 is on the composite layer 2. The sample collection pad 21 is lap-jointed at an upper surface of one end of the colloidal gold conjugate pad 22, the other end of the colloidal gold conjugate pad 22 is lap-jointed at an upper surface of one end of the color developing membrane 23, and the sample absorption pad 24 is lap-jointed at an upper surface of the other end of the color developing membrane 23. An end, closer to the colloidal gold conjugate pad 22, of an upper surface of the color developing membrane 23 is provided with a test line T 231, and an end, farther from the colloidal gold conjugate pad 22, of the upper surface of the color developing membrane 23 is provided with a control line C 232.

A thickness of the colloidal gold conjugate pad 22 and a thickness of the color developing membrane 23 are equal, and the width is named A. A width of the sample collection pad 21 and a thickness of the sample absorption pad 24 are equal, and this width is named B. The width of the sample collection pad 21 and the width of the sample absorption pad 24 are 2-6 times as great as the widths of the colloidal gold conjugate pad 22 and the color developing membrane 23. That is, B is 2-6 times as great as A. A length of the sample collection pad 21 is less than a length of the sample absorption pad 24. The composite layer 2 is I-shaped, the sample absorption pad 24 makes up an upper horizontal part, the colloidal gold conjugate pad 22 and the color developing membrane 23 jointly make up a middle vertical part, and the sample collection pad 21 makes up a lower horizontal part. The waterproof top layer 1 does not cover the sample collection pad 21 and completely covers the colloidal gold conjugate pad 22, the color developing membrane 23, and the sample absorption pad 24.

In a specific implementation process of the present disclosure, an overall thickness of the test strip is 0.2 mm-1.2 mm, preferably 0.3 mm-0.6 mm. After a lap-joint, an overall length of the test strip is 3 cm-8 cm, and an overall width of the test strip is 0.5 cm-2 cm.

In a specific implementation process of the present disclosure, materials of the waterproof top layer 1 and waterproof bottom layer 3 are made from polyethylene terephthalate (PET) waterproof transparent membranes with single-sided glue coating. A thickness of the waterproof top layer 1 and a thickness of the waterproof bottom layer 3 are 0.03 mm-0.06 mm.

In a specific implementation process of the present disclosure, lengths, lap-jointed with an upper surface of the color developing membrane 23, of the colloidal gold conjugate pad 22 and the sample absorption pad 24 are 1 mm-3 mm, and a length, lap-jointed with an upper surface of the colloidal gold conjugate pad 22, of the sample collection pad 21 is 1 mm-2 mm.

In a specific implementation process of the present disclosure, materials of the sample collection pad 21 and the sample absorption pad 24 are absorbent materials, and the material of the sample collection pad 21 is any absorbent material including cellulose, fiberglass, and a polyester fiber. The material of the sample absorption pad is any absorbent material including the cellulose, the fiberglass, and the polyester fiber. A thickness of the sample collection pad 21 and a thickness of the sample absorption pad 24 are <1 mm, more preferably <0.2 mm. The width of the sample collection pad 21 and the width of the sample absorption pad 24 are 0.5 cm-2 cm. The length of the sample collection pad 21 is 0.3 cm-0.9 cm, preferably 0.6 cm-0.8 cm. A water absorption capacity of the sample collection pad 21 is 200 g/m²-250 g/m². The length of the sample absorption pad 24 is 1 cm-1.5 cm. A grammage of the sample absorption pad 24 is 20 g/m²-80 g/m². A water absorption capacity of the sample absorption pad 24 is 400 g/m²-500 g/m².

In a specific implementation process of the present disclosure, a material of the color developing membrane 23 includes nitrocellulose or polyvinylidene fluoride (PVDF), preferably the nitrocellulose membrane. The material of the color developing membrane 23 is a porous material capable of uniformly and well adsorb protein. A material of the colloidal gold conjugate pad 22 is a polyester fiber membrane, and a grammage of the colloidal gold conjugate pad 22 is 60 g/m²-80 g/m². The width of the colloidal gold conjugate pad 22 and the width of the color developing membrane 23 are equal and 0.2 cm-0.4 cm. The length of the colloidal gold conjugate pad 22 is 0.2 cm-0.8 cm, and the thickness of the colloidal gold conjugate pad 22 is 0.1 mm-0.5 mm, preferably 0.13 mm-0.19 mm. The length of the color developing membrane 23 is 1.5 cm-2.5 cm, and the thickness of the color developing membrane 23 is 0.1 mm to 0.5 mm.

In a specific embodiment of the present disclosure, according to a tested disease type, an antibody conjugated to the colloidal gold on the colloidal gold conjugate pad 22 includes one or more of a mouse anti-β-human chorionic gonadotropin (HCG) monoclonal antibody, and monoclonal antibodies against luteinizing hormone (LH), gonorrhea streptococcus, Chlamydia trachomatis, Ureaplasma urealyticum, human papilloma virus, and herpes simplex virus. The color developing membrane 23 may be coated with one or more rabbit anti-β-HCG polyclonal antibody and polyclonal antibodies against LH, gonorrhea streptococcus, Chlamydia trachomatis, Ureaplasma urealyticum, human papilloma virus, and herpes simplex virus.

In a specific embodiment of the present disclosure, a diameter of the colloidal gold used for the colloidal gold conjugate pad 22 is 10 nm-200 nm.

The present disclosure further provides a method for manufacturing the test strip in the solution described above. The method includes:

A color developing membrane 23 is connected to a middle of a waterproof bottom layer 3, then a colloidal gold conjugate pad 22 and a sample absorption pad 24 are lap-jointed at two ends of the color developing membrane 23 respectively, and the colloidal gold conjugate pad 22 and the sample absorption pad 24 partially cover the color developing membrane 23.

A sample collection pad 21 is lap-jointed at the other end of the colloidal gold conjugate pad 22, and the sample collection pad 21 partially covers the colloidal gold conjugate pad 22.

A waterproof top layer 1 is connected, and the waterproof top layer 1 does not cover the sample collection pad 21 and completely covers the colloidal gold conjugate pad 22, the color developing membrane 23, and the sample absorption pad 24.

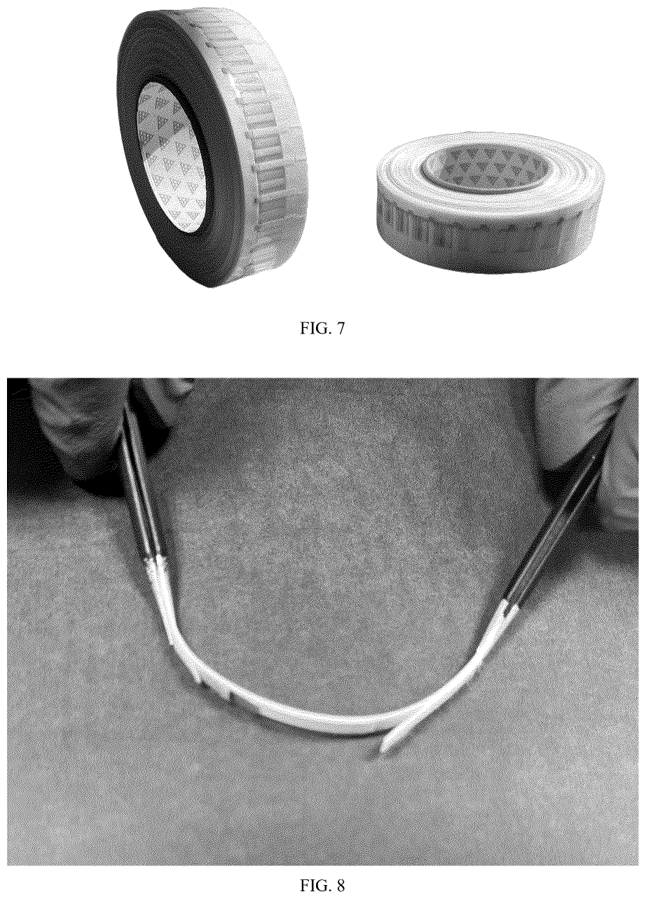

The present disclosure further provides the test strip roll in the solution described above. The test strips are connected mutually to form an elongated structure. In a specific processing process, the elongated structure may be rolled into a roll as shown in FIG. 6 and FIG. 7.

The present disclosure further provide a method for processing the test strip roll in the solution described above. A plurality of rollers are adopted to mount the rolls of waterproof top layers 1, sample collection pads 21, sample absorption pads 24, and waterproof bottom layers 3. The rolls are laid in the following method:
I. The colloidal gold conjugate pads 22 are bonded to one ends of the color developing membrane 23 to form middle vertical parts.
II. The middle vertical parts are bonded at equal distance in a width direction of the waterproof bottom layer 3 of the roll.
III. The sample collection pad 21 and the sample absorption pad 24 are dragged to two ends of the middle vertical part and bonded to the waterproof bottom layer 3.
IV. The waterproof top layer 1 is dragged to an uppermost position, and the waterproof top layer 1 does not cover the sample collection pad 21 and completely covers the colloidal gold conjugate pad 22, the color developing membrane 23, and the sample absorption pad 24.

The layers are tightly pressed with a pressing roller, and then wound with a winding roller to form the test strip roll.

The present disclosure further provides a method for using the test strip in the solution described above. The method includes:
the test strip on the test strip roll is cut, and the test strip is applied independently; or
the test strip is used after being connected to a sanitary product; or
the test strip is fixed under a surface layer of the sanitary product for use.

The present disclosure further provides a sanitary product. An upper surface of a surface layer or a lower part of the surface layer of the sanitary product is connected to or fixedly provided with the test strip in the solution described above.

In the present disclosure, a waterproof top layer 1 of the test strip is arranged upwards.

In a specific implementation process of the present disclosure, a lower part of the surface layer is the lower layer of the surface layer. In the present disclosure, the surface layer is made of a transparent material, and a test result may be directly viewed through the surface layer. A position of the test strip is preferably arranged in a way that the sample collection pad 21 is arranged under the vaginal orifice.

In a specific implementation process of the present disclosure, the sanitary product includes a sanitary napkin, a sanitary pad, a disposable period underwear, or a diaper. In the present disclosure, the sanitary product for women during a period includes the sanitary napkin or the sanitary pad. It should be noted that when the thin test strip is used in the sanitary product in contact with human body, a material of the sample collection pad 21 and a material of the sample absorption pad 24 include cellulose or polyester fiber.

The present disclosure further provides an application of the test strip in the solution described above to a sanitary product. The sanitary product in the present disclosure includes a sanitary napkin, a sanitary pad, a disposable period underwear, a diaper, and an incontinence pad.

The present disclosure further provides an application of the test strip or the sanitary product in the solution described above to manufacture of a product for monitoring a physiological indicator.

In the present disclosure, the testing a physiological indicator includes early pregnancy monitoring, ovulation period monitoring, or sexually transmitted disease monitoring.

The test strip or the sanitary product of the present disclosure may be used for health monitoring.

The present disclosure further provides an application of the test strip in the solution described above to a pet sanitary product. The pet sanitary product includes an incontinence pad, a diaper for the pet, and a paper diaper for the pet. For example, after the pet gets aged, the incontinence pad with the test strip may be used for the pet, and urinary incontinence of the pet can be automatically tested.

To further describe the present disclosure, the test strip, the sanitary product, and the application of the test strip provided by the present disclosure are described in detail below with reference to the accompanying drawings and in conjunction with the examples, which cannot be understood as limitation to the protection scope of the present disclosure.

### Example 1

As shown in FIG. 1-FIG. 2, the present disclosure provides an early pregnancy monitoring test strip for a sanitary product. The early pregnancy monitoring test strip includes a waterproof top layer 1, a composite layer 2, and a waterproof bottom layer 3 in sequence from top to bottom. The composite layer 2 is composed of a sample collection pad 21, a colloidal gold conjugate pad 22, a color developing membrane 23, and a sample absorption pad 24. Lengths, lap-jointed with an upper surface of the color developing membrane 23, of the colloidal gold conjugate pad 22 and the sample absorption pad 24 are 1.5 mm, and a length, lap-jointed with an upper surface of the colloidal gold conjugate pad 22, of the sample collection pad 21 is 1.5 mm. The waterproof top layer 1 is on the composite layer 2, the waterproof top layer 1 does not cover the sample collection pad 21 and completely covers the colloidal gold conjugate pad 22, the color developing membrane 23, and the sample absorption pad 24. A test line T and a control line C are arranged on the color developing membrane 23.

The waterproof top layer 1 and the waterproof bottom layer 3 are made from transparent PET membranes with a thickness of 0.03 mm.

A width of the sample collection pad 21 and a width of the sample absorption pad 24 are equal and are 1.5 cm. A material of the sample collection pad 21 is a polyester fiber membrane, and the sample collection pad 21 has a length of 0.7 cm, a thickness of 0.1 mm and a water absorption capacity of 200 g/m². The sample absorption pad 24 has a length of 1.3 cm, a thickness of 0.1 mm, a grammage of 40 g/m², and a water absorption capacity of 500 g/m².

A width of the colloidal gold conjugate pad 22 and a width of the color developing membrane 23 are equal and are 0.3 cm. The colloidal gold conjugate pad 22 has a length of 0.5 cm and a thickness of 0.2 mm. The color developing membrane 23 is a nitrocellulose membrane and has a length of 2 cm and a thickness of 0.2 mm.

A compound of colloidal gold and an anti-β-HCG mouse monoclonal antibody are provided on the colloidal gold conjugate pad 22, and a diameter of colloidal gold used is 20 nm.

The upper surface of the color developing membrane 23 is coated with a rabbit anti-β-HCG polyclonal antibody test line T 231 and a goat anti-mouse IgG antibody control line C line 232. An end, 0.7 cm away from the colloidal gold conjugate pad 22, of the upper surface of the color developing membrane 23 is provided with the test line T 231, and an end, 0.8 cm away from the sample absorption pad 24, of the upper surface of the color developing membrane is provided with the control line C 232.

An overall length of the test strip is 4 cm, and a thickness of a thickest position of the test strip is 0.4 mm.

A specific use method and function of this example are as follows: the test strip can be used separately or after being connected to the sanitary product. Female vaginal secretion or urine passes through the sample collection pad 21, moves into the colloidal gold conjugate pad 22 under the capillary siphon effect so as to be conjugated to colloidal gold coated with the mouse anti-β-HCG monoclonal antibody, then moves to the color developing membrane 23, and passes through the test line 231 and the control line 232 in sequence. When the sample solution is conjugated to the rabbit anti-β-HCG polyclonal antibody in the test line 231, the test line develops color. When the sample solution is conjugated to the goat anti-mouse IgG antibody in the control line 232, the control line 232 develops color. Whether the sample includes a high concentration of HCG can be identified by determining whether the test line 231 and the control line 232 are highlighted. High convenience is achieved, and the early pregnancy can be tested conveniently.

When used after being connected to the sanitary product, the test strip is provided with the sample collection pad 21 and the sample absorption pad 24 that have increased areas but are thinner, such that attachment of the test strip to the sanitary product can be improved, and the problems of high rigidity and the strong foreign body sensation caused by great thicknesses of a sample collection pad 21 and a sample absorption pad 24 of a common test strip can be solved. In addition, the sample collection pad 21 having the increased area improves a capture rate of liquid samples by the sample collection pad 21, and invalid and false negative test results are reduced advantageously. In addition, the test strip is different from the traditional colloidal gold test strip in that the rigid plastic plate at the bottom is removed, and the test strip can be rolled advantageously. The processing efficiency of a body is improved, and subsequent efficiency of processing the test strip to the sanitary product is further improved.

### Example 2

This example is different from Example 1 in that lengths, lap-jointed with an upper surface of a color developing membrane 23, of a colloidal gold conjugate pad 22 and a sample absorption pad 24 are 3 mm, and a length, lap-jointed with an upper surface of the colloidal gold conjugate pad 22, of a sample collection pad 21 is 2 mm. A maximum length, lap-jointed with the upper surface of the colloidal gold conjugate pad, of the sample collection pad is less than the lengths, lap-jointed with the upper surface of the color developing membrane, of the colloidal gold conjugate pad and the sample absorption pad. An objective of this design is to maximize sensitivity and specificity while ensuring a test speed. This technology is especially suitable for a case of a small amount of samples, the sample on the sample collection pad can be rapidly transferred, the sample on the color developing membrane can further fully react, and the overall test accuracy can be further improved.

### Example 3

This example is identical to Example 1 in structures of parts except that an antibody conjugated to colloidal gold on a colloidal gold conjugate pad 22 is a mouse anti-LH monoclonal antibody, and an antibody coating the T line on the color developing membrane 23 is a rabbit anti-LH polyclonal antibody. The test strip is used to monitor an ovulation period.

A specific use method and function of this example are as follows: the test strip can be used separately or after being connected to the sanitary product. Female vaginal secretion or urine passes through a sample collection pad 21, moves into the colloidal gold conjugate pad 22 under a capillary siphon effect so as to be combined with colloidal gold coated with the antibody, then moves to the color developing membrane 23, and passes through a test line 231 and a control line 232 in sequence. When a sample solution is combined with the polyclonal antibody in the test line 231, the test line develops color. When the sample solution is combined with the goat anti-mouse IgG antibody in the control line 232, the control line 232 develops color. A subject starts a test on a first day of menstruation, and performs the test once by selecting fixed time every day. When the test line 231 and the control line 232 develop color, an ovulation period is indicated, and a best pregnancy period can be determined.

When used after being connected to the sanitary product, the test strip is provided with the sample collection pad and a sample absorption pad that have increased areas but are thinner, such that attachment of the test strip to the sanitary product can be improved, and the problems of high rigidity and the strong foreign body sensation caused by great thicknesses of a sample collection pad and a sample absorption pad of a common I-shaped test strip can be solved. In addition, the sample collection pad having the increased area improves a capture rate of liquid samples by the sample collection pad, and invalid and false negative test results are reduced advantageously.

### Example 4

This example is identical to Example 1 in structures of parts except that a test strip is used to test a sexually transmitted disease. According to a disease type, an antibody conjugated to colloidal gold on a colloidal gold conjugate pad includes one or more of a mouse antibody, and monoclonal antibodies against gonorrhea streptococcus, Chlamydia trachomatis, Ureaplasma urealyticum, human papilloma virus, and herpes simplex virus. Correspondingly, an antibody coating a T line of a color developing membrane may be one or more of a rabbit antibody and polyclonal antibodies against gonorrhea streptococcus, Chlamydia trachomatis, Ureaplasma urealyticum, human papilloma virus, and herpes simplex virus.

A specific use method and function of this example are as follows: the test strip can be used separately or after being connected to the sanitary product. Female vaginal secretion or urine passes through a sample collection pad 21, moves into the colloidal gold conjugate pad 22 under the capillary siphon effect so as to be conjugated to colloidal gold coated with the antibody, then moves to the color developing membrane 23, and passes through the test line 231 and a control line 232 in sequence. When a sample solution is conjugated to the polyclonal antibody in the test line 231, the test line develops color. When the sample solution is combined with the goat anti-mouse IgG antibody in the control line 232, the control line 232 develops color. Whether the sample includes pathogenic bacteria of a sexually transmitted disease can be identified by determining whether the test line 231 and the control line 232 are highlighted. High convenience is achieved, and the sexually transmitted disease can be tested primarily.

When used after being connected to the sanitary product, the test strip is provided with the sample collection pad and a sample absorption pad that have increased areas but are thinner, such that attachment of the test strip to the sanitary product can be improved, and the problems of high rigidity and the strong foreign body sensation caused by great thicknesses of a sample collection pad and a sample absorption pad of a common test strip can be solved. In addition, the sample collection pad having the increased area improves a capture rate of liquid samples by the sample collection pad, and invalid and false negative test results are reduced advantageously. In addition, the test strip is different from the traditional colloidal gold test strip in that the rigid plastic plate at the bottom is removed, and the test strip can be rolled advantageously. The processing efficiency of a body is improved, and subsequent efficiency of processing the test strip to the sanitary product is further improved.

### Experimental Example 1: A Bending Experiment

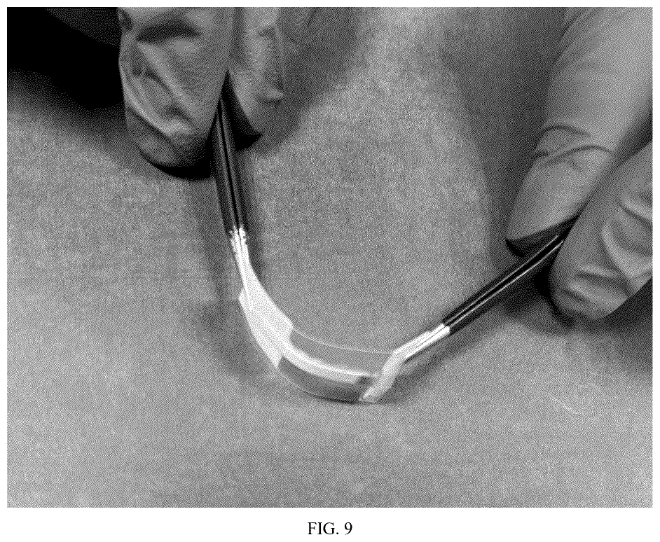

As shown in FIG. 8, tweezers were adopted to clamp ends of a sample collection pad 21 and a sample absorption pad 24 of a traditional I-shaped colloidal gold test strip, and bent the test strip. It was found that a colloidal gold conjugate pad was separated from a color developing membrane, and the sample absorption pad was also separated from the color developing membrane. It can be seen that if the traditional colloidal gold test strip is narrowed into a thinner I-shaped structure for reducing a foreign body sensation of the traditional colloidal gold test strip, overall test accuracy can be lower. The reason is that after a wide and thick test strip is directly narrowed, since a bottom is a polyvinyl chloride (PVC) plastic bottom plate, the test strip is likely to bend and deform due to human movement during use, and is hard to restore due to its hard structure. As a result, the sample is hindered from evenly spreading forwards on the parts in the bent zone, and the test accuracy is still affected.

As shown in FIG. 9, the inventor clamped ends of a sample collection pad 21 and a sample absorption pad 24 of a thin test strip in Example 1 of the technical solution with tweezers, and bent the test strip. It was found that neither of a colloidal gold conjugate pad 22 and the sample absorption pad 24 was separated from a color developing membrane 23.

### Experimental Example 2: A Waterproof Test

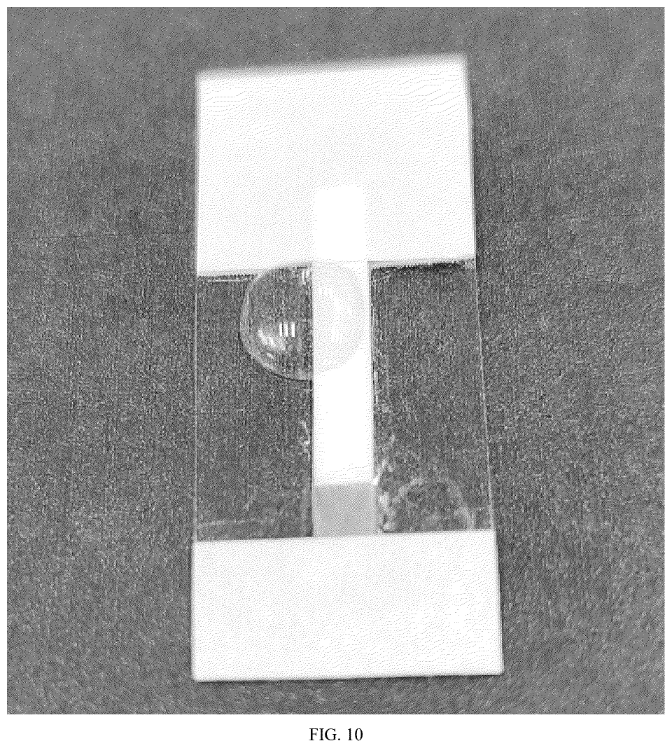

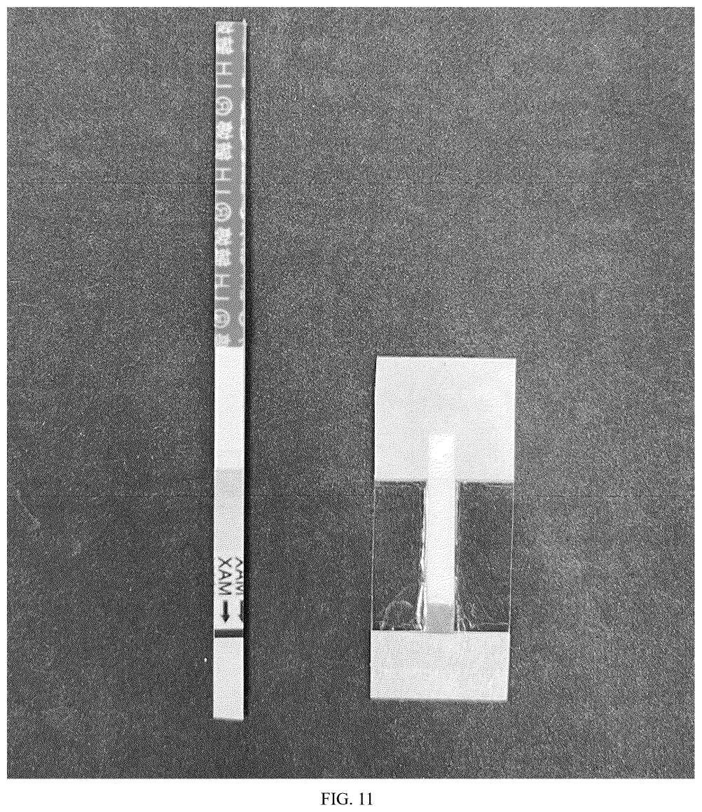

As shown in FIG. 10, the inventor dropped water on the waterproof top layer 1 of the thin test strip in Example 1 of this technical solution, and left the test strip standing for 2 hours. It can be seen that water drops failed to permeate. Based on this, the waterproof top layer 1 and waterproof bottom layer 3 of this technology protect the overall test strip to form a double-sided hydrophobic material. Moreover, the waterproof top layer 1 and the waterproof bottom layer 3 seal and protect the composite layer 2, thus prolonging an overall service life and ensures test accuracy.

### Experimental Example 3: Comparison of Sample Injection Areas

As shown in FIG. 11, the sample injection area of a colloidal gold test strip in an I-shaped structure is obviously narrow. However, the sample injection area of the thin test strips formed by this technical solution is obviously wider. This means that a contact area of this technology with a sample is larger, tests are possible especially in a case of a small number of samples, applicable scenarios are expanded, and test accuracy is further improved.

### Experimental Example 4: A Humidity Test

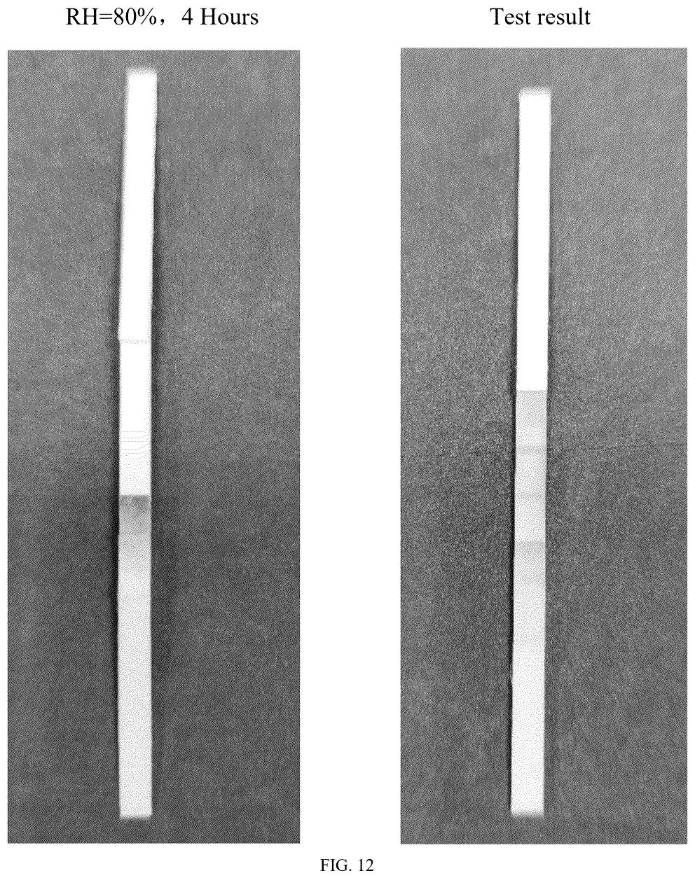

As shown in FIG. 12, a traditional colloidal gold test strip in an I-shaped structure was placed in a high humidity environment with RH=80% for 4 hours. A “colorful” part on a left side in FIG. 12 indicated colloidal gold, and it can be clearly seen that distribution of the colloidal gold was uneven. On a sample collection pad of the colloidal gold test strip on the left side in FIG. 12, 50 microliters of luteinizing hormone solutions with a concentration of 25 mmol/L was dripped, and the colloidal gold test strip on the right side in FIG. 12 was obtained after 10 minutes. It can be clearly seen that a bleaching effect of the color developing membrane is poor and the strip is not obvious.

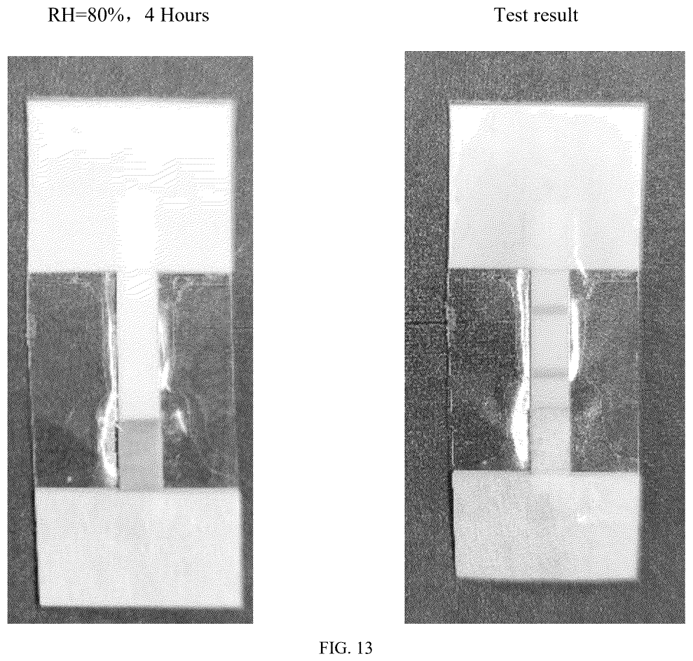

As shown in FIG. 13, the thin test strip of Example 1 of this technical solution was placed in a high humidity environment with RH=80% for 4 hours. It can be seen from a left side of the figure, colloidal gold was evenly and stably distributed. On a sample collection pad 21 of the thin test strip on the left side in FIG. 13, 50 microliters of luteinizing hormone solutions with a concentration of 25 mmol/L was dripped, and a picture on the right side in FIG. 13 was obtained after 10 minutes. It can be clearly seen that a color developing membrane is obviously bleached, and strips of the test line T and the control line C are clear.

To sum up, the thin test strip of this technology is designed by combining double-sided transparent membranes and the I-shaped composite layer, thus solving the problem of the foreign body sensation, and further solving the problem that the human movement bends the test strip and reduces the test accuracy.

The examples above are merely the examples of the present disclosure, and common general knowledge including specific technical solutions and/or features in the solution is not described herein. It should be noted that for those skilled in the art, several modifications and improvements can be made without departing from the technical solution of the present disclosure, and such modifications and improvements should also be considered to fall within the protection scope of the present disclosure without influence on implementation effects of the present disclosure and practicability of the patent. The protection scope required by the present application should be subject to the contents of the claims, and explanation of the specific embodiments, etc. in the description can be used to explain the contents of the claims.

What is claimed is:

1. A thin test strip, comprising: a waterproof top layer, a composite layer, and a waterproof bottom layer in sequence from top to bottom, wherein the composite layer comprises a sample collection pad, a colloidal gold conjugate pad, a color developing membrane, and a sample absorption pad, the waterproof top layer does not cover the sample collection pad and completely covers the colloidal gold conjugate pad, the color developing membrane, and the sample absorption pad, and the composite layer is I-shaped.

2. The thin test strip according to claim 1, wherein the waterproof top layer and the waterproof bottom layer are made from polyethylene terephthalate (PET) waterproof transparent membranes.

3. The thin test strip according to claim 2, wherein a thickness of the waterproof top layer and a thickness of the waterproof bottom layer are 0.03 mm-0.06 mm.

4. The thin test strip according to claim 1, wherein a length of the sample collection pad is less than a length of the sample absorption pad.

5. The thin test strip according to claim 4, wherein the length of the sample collection pad is 0.3 cm-0.9 cm, and the length of the sample absorption pad is 1.0 cm-1.5 cm.

6. The thin test strip according to claim 5, wherein a width of the sample collection pad and a width of the sample absorption pad are 0.5 cm-2 cm.

7. The thin test strip according to claim 6, wherein the width of the sample collection pad and the width of the sample absorption pad are equal.

8. The thin test strip according to claim 7, wherein a thickness of the sample collection pad and a thickness of the sample absorption pad are <1 mm.

9. The thin test strip according to claim 1, wherein a length of the colloidal gold conjugate pad is 0.2 cm-0.8 cm.

10. The thin test strip according to claim 9, wherein a width of the colloidal gold conjugate pad and a width of the color developing membrane are equal.

11. The thin test strip according to claim 10, wherein the width of the colloidal gold conjugate pad and the width of the color developing membrane are 0.2 cm-0.4 cm.

12. The thin test strip according to claim 11, wherein a thickness of the colloidal gold conjugate pad is 0.1 mm-0.5 mm.

13. The thin test strip according to claim 1, wherein a length of the color developing membrane is 1.5 cm-2.5 cm.

14. The thin test strip according to claim 13, wherein a thickness of the color developing membrane is 0.1 mm-0.5 mm.

15. The thin test strip according to claim 1, wherein lengths, lap-jointed with an upper surface of the color developing membrane, of the colloidal gold conjugate pad and the sample absorption pad are 1 mm-3 mm, and a length, lap-jointed with an upper surface of the colloidal gold conjugate pad, of the sample collection pad is 1 mm-2 mm.

16. The thin test strip according to claim 1, wherein a material of the sample collection pad is any absorbent material comprising cellulose, fiberglass, and a polyester fiber, and a material of the sample absorption pad is any absorbent material comprising the cellulose, the fiberglass, and the polyester fiber.

17. The thin test strip according to claim 1, wherein a material of the color developing membrane is any porous material comprising nitrocellulose or polyvinylidene fluoride, an end, closer to the colloidal gold conjugate pad, of an upper surface of the color developing membrane is provided with a test line T, and an end, farther from the colloidal gold conjugate pad, of the upper surface of the color developing membrane is provided with a control line C.

18. The thin test strip according to claim 1, wherein a thickness of the test strip is 0.2 mm-1.2 mm, a length of the paper strip is 3 cm-8 cm, and a width of the test strip is 0.5 cm-2 cm.

19. A test strip roll, comprising a roll formed by mutually connecting the thin test strips according to claim 1.

20. An application of the thin test strip according to claim 1 to a sanitary product, manufacture of a product for monitoring a physiological indicator, or a pet sanitary product.
